(19) United States
(12) Patent Application Publication
(10) Pub. No.: US 2023/0414196 A1
(43) Pub. Date: Dec. 28, 2023

(54) SYSTEM FOR PERFORMING AN AUTOMATED ULTRASONIC SCAN OF THE EYE

(71) Applicant: MIKAJAKI, SA, Plan-les-Ouates (CH)

(72) Inventors: Joel HOCHNER, Modiin (IL); Doron SMULIAN, Haifa (IL); Ofir SHUKRON, Paris (FR); Michael ASSOULINE, Paris (FR); Nir SINAI, Alon HaGalil (IL); Patricia KOSKAS, Paris (FR)

(73) Assignee: MIKAJAKI, SA, Plan-les-Ouates (CH)

(21) Appl. No.: 18/037,502

(22) PCT Filed: Nov. 18, 2021
(86) PCT No.: PCT/IL2021/051380

§ 371 Date: May 17, 2023

(30) Foreign Application Priority Data
Nov. 18, 2020 (IL) 278818

(51) Int. Cl. A61B 8/10; A61B 8/00; A61B 8/08

(52) U.S. Cl. CPC A61B 8/10; A61B 8/4461; A61B 8/4227; A61B 8/0858

(57) ABSTRACT

A system that is capable of performing a fully automated ultrasonic (US) scan. The subject that is undergoing the scan associates with the system and the scan is performed without any human intervention. The US probe comprises an US transducer that is configured to generate US signals and detect their reflections, to collect acoustic data during the scan. The US probe is moved by a probe-motioning unit that is affixed and is configured to move the US probe in up to six degrees of freedom according to the desired scan pattern that is determined by a processing circuitry of the system, i.e. a control unit. Therefore, any anatomical landmark of the eye can be scanned from any desired angle. The processing circuitry is configured to control the operation of the US probe and the probe-motioning unit to execute a desired scan pattern.

### TECHNOLOGICAL FIELD

The present disclosure is in the field of eye examination devices, in particular ultrasonic devices.

### BACKGROUND ART

References considered to be relevant as background to the presently disclosed subject matter are listed below:
WO 2019/172232
WO 01/49223
U.S. Pat. No. 8,496,588
JP 2015104498
JP 4342638

Acknowledgement of the above references herein is not to be inferred as meaning that these are in any way relevant to the patentability of the presently disclosed subject matter.

### BACKGROUND

Ophthalmic ultrasound (OUS, ocular echography or ocular B-scan) is a non-invasive procedure routinely used in specialized clinical practice to assess the structural integrity and pathology of the eye.

OUS is carried out by using high or low ultrasonic frequencies. High frequencies probes (10, 20 or 50 MHz) are used in ocular echography to obtain an image with greater resolution than the low frequencies. The use of higher frequencies probes implies poorer tissue penetration (shorter depth of field) and generate heat that may alter the delicate tissue if excessive energy is delivered in too short a time.

For example, standard 10 MHz probes allow enough penetration to properly examine the delicate ocular structures of the entire globe, while 20 MHz and 50 MHz are dedicated to a more detailed analysis of the more superficial aspects (anterior segment, iris, irido-corneal angle and crystalline lens).

Most of the OUS are performed in two types of scans—A-scan and B-scan. The A-scan provides a simple unique line of echoes reflected by the internal structures of the eye, to measure the main distances between cornea, lens and retina (ocular biometry). The B-scan moves the transducer along a given plane to create a two-dimensional image from a very thin slice of tissue oriented perpendicular to the cylinder of the probe.

Ultrasound images can be obtained through the patient's eyelids or with the probe directly on the surface of the eye with appropriate topical anesthesia, which improves resolution.

The entire globe can be analyzed as a step wise process to explore four dynamic quadrant views and one more static slice through the macula and optic disc (longitudinal macula LMAC).

An OUS provides a very detailed presentation of the eye status and can be used for examination and diagnostic of eye conditions to thereby provide a suitable treatment or prevention treatment for the examined subject.

An OUS examination depends on the expertise of the practitioner and is not always accessible to patients. Therefore, there is a need to provide a convenient and accessible system that can carry out an OUS in an automated or semi-automated process

### General Description

The present disclosure discloses a system that is capable of performing a fully automated ultrasonic (US) scan. The subject that is undergoing the scan associates with the system and the scan is performed without any human intervention. The association between the subject and the system is either by fixed association, such as an adjustable strap that fastens the head of the subject, or part of the head, to the system; or by engagement of the head, or part of the head, with a portion of the system such that at least the examined eye is suitably positioned with respect to an US probe of the system.

The US probe comprises an US transducer that is configured to generate US signals and detect their reflections, to collect acoustic data during the scan. The US probe is moved by a probe-motioning unit that is affixed and is configured to move the US probe in up to six degrees of freedom according to the desired scan pattern that is determined by a processing circuitry of the system, i.e. a control unit. Therefore, any anatomical landmark of the eye can be scanned from any desired angle.

US scan pattern refers to the position and orientation of the US probe with respect to the eye and to the mode of operation of the US probe, namely the gain intensity and/or frequency of the US signal. It is to be understood that acoustic data is referred to any acoustic data that is obtained by US scan, e.g. A or B scans, as well as doppler signals indicative of the velocity of blood within the intraocular and optic nerve vasculature and it includes image and/or doppler data that is reconstructed by US scans.

The processing circuitry is configured to control the operation of the US probe and the probe-motioning unit to execute a desired scan pattern, i.e. spatial pattern of the probe and operational parameters of the US transducer, e.g. scanning windows, gain, etc.

The processing circuitry is configured to execute either (i) a default, pre-set scan pattern that is configured to collect basic acoustic data of the eye; or (ii) a planned scan that is based on specific characteristics of the subject. The planned scan may follow a first, pre-set scan, in which an initial first acoustic data is collected and based on the findings of this first scan the second scan is planned and executed to collect additional data that is required for the determination of at least one condition of the eye of the subject. It is to be noted that a condition of the eye is either pathology, disease or any abnormality of the eye.

Thus, an aspect of the present disclosure provides a system for performing an automated US scan of an eye of a subject. The system includes an US transducer probe configured for generating ultrasonic signals and detecting reflections thereof. The system further includes a placement arrangement for allowing positioning of the eye of the subject at a desired position with respect to the US probe. The placement arrangement can be a shaped structure to position and stabilize the head or part of the head, a dedicated headrest, or a fixation arrangement for fixing the device onto the head of the patient at a desired orientation with respect to the US probe.

A probe-motioning unit is configured to allow movement of the probe in at least three degrees of freedom or more, e.g. four or five degrees of freedom. Typically, for obtaining the maximal flexibility, the probe-motioning unit is configured to allow movement of the probe in six degrees of freedom, namely the probe is capable to move along three axes and rotate about each one of them.

A processing circuitry configured for controlling the movement of the probe-motioning unit and the operation of the US probe to execute at least a first US scan pattern through the eyelid of the subject or directly from the eye surface to collect at least a first US acoustic data indicative of one or more eye conditions of the subject.

In some embodiments of the system, the first US scan pattern is a pre-set US scan pattern, namely a scan with pre-defined parameters of at least one of: positioning, orientation, mode of operation of the US probe or any combination thereof.

In some embodiments of the system, the processing circuitry is configured to: (i) analyze said first US acoustic data to identify one or more regions of interest, i.e. anatomic landmarks or parts of the eye, potential abnormalities, or any other diagnostic features indicative of a specific eye condition, etc.; (ii) plan a second US scan pattern, based on the first US acoustic data, and control the probe-motioning unit and the US probe for executing said second US scan for collecting a second US acoustic data indicative of said regions of interest. The second US scan provides additional US scanning parameters with respect to these regions of interest including, but not limited to, different scanning angle of the probe, different US gain, different frequencies, different focusing distance, different spatial sectioning in order to improve the acquisition of signal and identification of diagnostic features.

In some embodiments of the system, the processing circuitry is configured to analyze said first US acoustic data and identify a predefined landmark of the eye, such as the optical nerve of the examined eye. The optical nerve is used as a reference landmark for image analysis of the US acoustic data. For example, the gaze direction of the examined eye can be derived by the position of the optical nerve. Thus, the processing circuitry is configured to validate that the eye is gazing to the required direction during the first and/or second scan by recognizing the position of certain landmarks of the eye related to the position, shape, thickness or relative distance of ocular and intraocular structures (ocular biometry), to their echogenicity or to the velocity of blood within the intraocular or the optic nerve vessels, spontaneously or as a result of calibrated pressure applied to the eye wall.

In some embodiments of the system, the processing circuitry is further configured for performing feature analysis, such as image analysis and/or applying machine learning algorithms, on said at least first and/or second US acoustic data to automatically identify one or more predefined conditions of the eye of the subject, namely a closed list of eye conditions that the processing circuitry is trained to identify, e.g. by machine learning process. The conditions can include pathological conditions or features that imply on a pathological condition. In some embodiments, a non-limiting list of conditions, i.e. eye conditions or pathologies of the eye that may be identified by the US scan is: Vitreous conditions such as Normal (VN), posterior detachment of the vitreous (VPD), to hemorrhage (VHE), endophthalmitis (VE), hyalitis (VHY); Retina conditions, such as Normal (RN), Retinal detachment total (RDT), partial (RDP), Attachment (RA), Retinoschisis (RS). Tumor retinoblastoma (RTR); Macula conditions, such as; Normal (MN), Age related degeneration (MARD), Drusen of macula (MD), edema (ME); Papilla conditions, such as Normal (PN), Excavated severity 1-3 (PE1, PE2, PE3), Calcified drusen (PD), Edema (PE), Tumor melanocytoma (PTM); Choroid conditions, such as Normal (CN), Detached; partial (CDP), total (CDT). Haemorrhagic detachment (CHD); Tumor conditions, such as Melanoma (CTM), Haematoma (CTH), Angioma (CTA), parasitic cyst (CTPC), Metastasis (CT2M); Scleral conditions, such as Normal (SN), Thickening (STK), Thinning (STN), Invasion (STI), Calcification (SC); Optic nerve conditions, such as Normal (ON), Atrophy (OA), Enlargement of perioptic spaces (OE), Tumor of perioptic spaces (OTP); Intraocular foreign body conditions, such as (IOFB); Post (after) surgical vitreous conditions, such as air/gas (ASG), silicon (ASS); or any other conditions (OTH).

In some embodiments of the system, the feature analysis includes scoring each of the predefined conditions, based on the findings of the feature analysis, and the identification of a condition is determined upon satisfying a certain score condition, e.g. exceeding a score threshold or being the highest score among the scores of any other conditions. Namely, the conditions are assumed to be identified in the scan if sufficient related data is recognized in the analysis of the data and the scoring of the condition satisfies a predetermined score. e.g. a score that is obtained by machine learning process.

In some embodiments of the system, said feature analysis of the acoustic data comprises automatic extraction of features for being used for further analysis, segmentation of the acoustic data into anatomical regions of interest, and/or scoring the frame for further usability if exceeding a selected threshold.

In some embodiments of the system, the processing circuitry is configured to analyze, segment and process the first and/or second US acoustic data. The first and/or second US acoustic data comprises A or B scan images.

The following is an example of an analysis process of US acoustic data, in particular the acoustic image data. The process includes pre-processing of each individual image, in the case of 2D scans, and image stack processing in the case of 3D US scans, where pixel positional information can be used across the image stack and between spatially adjacent 2D scans to improve results. Pre-processing is aimed at cleaning noise and artifacts from images. The process then includes applying techniques to automatically segment three key anatomical regions in each scan: the eye globe, the optic nerve, and the vitreous. Localization of these regions is performed using pyramid image processing in decreasing image resolution for each successive pyramid level. Decreasing resolutions in each pyramid level is performed by subsampling and image smoothing. Localization of the three anatomical regions in each pyramid image slice is performed using transformations to detect specific shapes (e.g. globes/spheres/cylinders, e.g. by the Hough transform), and is complemented by graph theoretic algorithms to refine the localization (boundary) of the vitreous and globe, for example, to a sub-pixel accuracy. In addition, pattern matching and geometric model fitting is applied along the detected globe in each given image resolution, to localize the optic nerve Geometric information about the sphericity of the segmented globe, the integrity (smoothness) of the vitreous and the visibility of the optic nerve is extracted and used for scoring frames for their quality and for frame selection for subsequent analysis. Frames are discarded from analysis in case of insufficient score. Once the three anatomical regions are localized in a given image resolution, the process further includes applying a trained multi-label machine-learning predictor to assign the most probable labels (classes) describing one or more possible pathologies in each of the three anatomical regions. Assigned labels in one anatomical region is used as a feature for another, to reinforce the machine learning's decision.

In some embodiments of the system, the processing circuitry is configured to extract from said at least first and/or second US acoustic data biometry parameters indicative of the eye structure of the subject, e.g. irido-corneal angle, the anterior chamber depth, the crystalline lens thickness, the axial length of the globe.

In some embodiments of the system, the processing circuitry is configured to control parameters of said US signals, e.g. signal intensity, frequency, signal generation windows, etc.

In some embodiments of the system, the processing circuitry is configured to perform real-time image analysis of said at least first and/or second acoustic data and real-time adjusting the respective US scan pattern, either the pre-set pattern or the second pattern, for collecting desired acoustic data.

In some embodiments of the system, the placement arrangement includes a fixation unit for fixedly association of the head of the subject, or part of the head, to the US probe.

In some embodiments of the system, the fixation unit includes fasteners for fastening the head of the unit.

In some embodiments, the system is portable and designed to fit over a head of the subject. For example, the system may be in the form of monocular or binocular that fits over the examined eye and/or the fellow eye.

In some embodiments of the system, the US probe is fixed to the probe-motioning unit and move in correlation with the movement of the probe-motioning unit or a part thereof, such as a platform where the US probe is mounted on.

In some embodiments of the system, the probe-motioning unit comprises a plurality of arms, each is fixed to the US probe at one end, and fixed to a support structure, such as a housing, of the system. Movement, extension, or retraction of each of the arms results in movement of the US probe. The sum of all the movements of the arms yields the eventual movement of the US probe. Each arm is driven by a motor that allows said movements to obtain that desired position and orientation of the US probe.

In some embodiments of the system, the probe-motioning unit comprises a platform and the US probe is mounted on said platform.

In some embodiments, the system includes a pressure sensor for measuring the applied pressure on the eyelid by the US probe. During the US scan, the probe applies a certain pressure on the eyelid of the eye to maintain in contact with the eye during the scan. Thus, the applied pressure is monitored and regulated by the pressure sensor and the processing circuitry that is in data communication with the pressure sensor to receive the data therefrom and control the movement of the US probe accordingly.

In some embodiments of the system, the processing circuitry is configured to receive the pressure measurement and adjust the applied pressure to be within a selected pressure range.

In some embodiments, the system includes a probe positioning sensor, e.g. a gyroscope-based sensor or any accelerometer, that is configured to monitor the spatial position of the US probe and transmit positioning data to the processing circuitry.

In some embodiments of the system, the processing circuitry is configured to process the positioning data for monitoring and/or adjusting the movement of the probe-motioning unit for executing the desired first or second US scan pattern.

In some embodiments, the system includes a fellow eye positioning and/or monitoring unit. The fellow eye positioning and/or monitoring unit comprises at least one of the following: (a) a light emitting device, e.g. a display, an array of light emitting sources such as LEDs or any other visualization means, to guide the movement of the fellow eye and use the conjugate movement of the examined eye, thereby resulting in an alignment of the examined eye along a pre-defined or desired position; (b) an optical sensor, i.e. an image sensor capable of capturing images or stream of images, to monitor the position of the fellow eye that is indicative of the position of the examined eye. Thus, the system may monitor the fellow eye status and derive the respective status of the examined eye therefrom. Furthermore, the system may provide guidance for the subject to gaze towards a selected direction with the fellow eye and by that obtain the desired spatial status of the examined eye for performing the US scan at the right conditions for collecting the desired acoustic data. The fellow eye positioning and/or monitoring unit is positioned in the system in a way that while the system is in use, namely examining the scanned eye, it is visible to the fellow eye. In other words, the fellow eye positioning and/or monitoring system is constantly in line of sight with the fellow eye, namely the eye that is not scanned, during scanning of the other eye.

The fellow eye is not the eye being scanned. Since the movement of the eyes are conjugated, by guiding the movement of the fellow eye, the scanned eye moving in correlation with the fellow eye according to a desired movement pattern directed by the movement guiding unit.

In some embodiments, the system includes an ocular movement guiding unit for guiding ocular movement of the subject. The ocular movement guiding is recognizable by the subject during the execution of the US scan pattern, either visually or audibly.

In some embodiments of the system, the ocular movement guiding unit comprises visual means for guiding the gaze of the fellow eye. The visual means can array of LEDs, a display, or any other optional guiding visual means. The visual means are disposed such that they are visible to the fellow eye during a scan of the other, scanned eye.

In some embodiments, the system is designed in the shape of glasses, the US probe is disposed in association with a first side of the glasses and the fellow eye positioning and/or monitoring unit and/or the ocular movement guiding unit are associated with a second side of the glasses. Thus, while one eye is scanned, the fellow eye is monitored to realize the ocular position of the scanned eye. It is to be noted that the glass shape of the system allows it to be fitted in two ways, each way is intended to scan a different eye (i.e., one way to scan the left eye and a second way to scan the right eye).

In some embodiments of the system, the ocular movement guiding unit includes a speaker device configured to produce audible guiding instructions for ocular movement of the subject. In some embodiments the speaker device is operable by the processing circuitry to produce audible guiding instructions in accordance with the requirements of the executed first or second US scan pattern. The speaker is configured to be heard by the subject during a scan of the system of the other scanned eye. For example, the system may output guiding instructions through the speaker to gaze towards a certain direction with the fellow eye.

In some embodiments of the device, the ocular movement guiding unit includes a pressure generating component for applying localized pressure on subsequent regions of the face of the subject thereby guiding the movement of the eye being scanned or the fellow eye.

In some embodiments of the system, the processing circuitry is configured to operate the ocular movement guiding unit in synchronization with the execution of any of the at least first and/or second US scan patterns, thereby obtaining synchronized movement of the scanned eye according to the scan stage. For example, if a certain US scan requires the eye to be at a certain spatial status, i.e. gazing towards a certain direction, the ocular movement guiding system may output guiding instructions for bringing the fellow eye, and therefore also the examined eye, to the desired spatial status. The system may also monitor the fellow eye to recognize that it is in the desired status and then executes the desired scan.

In some embodiments, the system includes a safety switch for allowing immediate stop of the operation of the system.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1B are block diagrams of non-limiting examples of the system according to embodiments of the present disclosure.

FIGS. 2A-2C are perspective views of schematic illustrations of a non-limiting example of an embodiment of the system according to an aspect of the present disclosure. FIG. 2A is a generally back view of a part of the system; FIG. 2B is a generally front view of a part of the system; FIG. 2C is a generally front view of the system showing the frame the fits over the wearers' face.

FIG. 3 is a schematic illustration of a top view of a non-limiting example of the eye positioning/monitoring unit of the system.

### DETAILED DESCRIPTION OF EMBODIMENTS

The following figures am provided to exemplify embodiments and realization of the invention of the present disclosure.

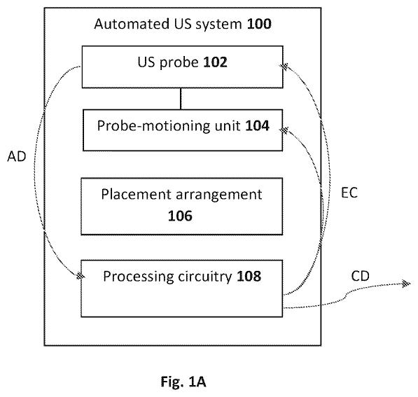

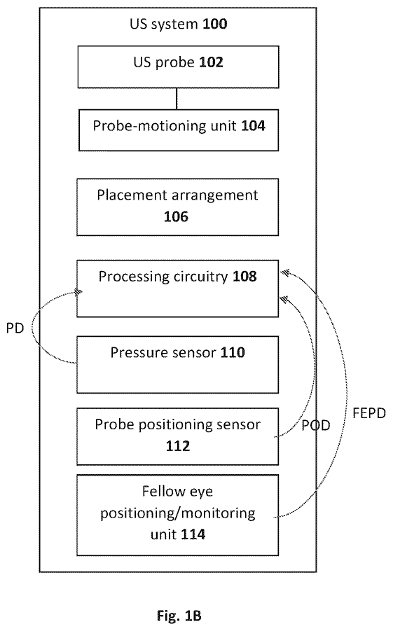

Reference is first being made to FIGS. 1A-1B, which are block diagrams of different non-limiting embodiments of the automated US system according to an aspect of the present disclosure. FIG. 1A exemplifies an automated US system 100 that includes a US transducer probe 102 (interchangeably referred to as “US probe” or “probe” throughout the application) and is associated or fixed to a probe-motioning unit 104. The probe-motioning unit 104 is configured to move the US probe according to a desired and selected pattern. The probe-motioning unit 104 is capable to move the probe 102 at three or more degrees of freedom. Preferably, the probe-motioning unit 104 is capable to move the probe 102 at six degrees of freedom, namely spatial movement along three orthogonal axes and rotate about each axis. By this capability, any desired scan of the eye can be taken. i.e. any anatomic part of the eye can be scanned from any desired angle.

The system 100 further includes a placement arrangement 106 that is configured for allowing the patient to place his/her head or part of the head against the system such that the examined eye is at the right desired position with respect to the US probe 102 to allow performing the US scan. The placement arrangement may be in the form of an adapted frame, a depression, a head rest, a fixation element, a strap, or any combination thereof.

A processing circuitry 108 is configured to control the US probe 102 and the probe-motioning unit 104 by execution commands EC to execute desired US scan pattern. A US scan pattern is a collection of acoustic data AD, i.e. ultrasonic image data from one or more locations of the eye in one or more orientations of the probe, namely one or more angles. The processing circuitry is further configured for receiving the acoustic data AD and analyze it to identify one or more conditions of the eye. The analysis may be carried out by any suitable image processing, e.g. by machine learning techniques. The processing circuitry is configured to output conditions data CD indicative of the findings of the conditions of the eye. The conditions data CD may be received by any output device, e.g. a display, a mobile device, a computer, etc.

FIG. 1B is a block diagram of another embodiment of the system of the present disclosure. FIG. 1B exemplifies a system similar to that of FIG. 1A having some additional elements. The system further includes a pressure sensor 110, probe-positioning sensor 112, fellow eye positioning/monitoring unit 114. It is to be noted that the system of FIG. 1A may include, in addition to the elements that are presented in the figure, any one of the added elements of FIG. 1B.

The pressure sensor 110 is configured for sensing the pressure that is applied on the eye by the probe 102 during the scan. The pressure data PD that is obtained by the pressure sensor 110 is transmitted to the processing circuitry 108 and the processing circuitry is configured to control the probe-motioning unit 104 to maintain the applied pressure of the probe 102 on the eye in a desired range of pressure levels, i.e. between a high-pressure limit and low-pressure limit. This range may be varied at different parts of the scan, namely when examining a first anatomical landmark, a first pressure level is selected within a first range is applied and when examining a second anatomical landmark, a second pressure level is selected within a second range is applied.

The probe-positioning sensor 112 is configured for sensing the position of the probe 102. The probe-positioning sensor may sense the position of the probe with respect to either manual initial position setting of the prove with respect to the examined eye or with respect to a landmark of the eye that is identified during the initial phase of the scan. For example, following the identification of the position of the optical nerve by the processing circuitry, the probe-positioning sensor is configured to provide a relative reference position from the optical nerve, including mechanical position of the probe, and the scan position with respect to the optical nerve. The positioning data POD that is obtained by the probe-positioning sensor 112 is transmitted to the processing circuitry 108 for monitoring the position and orientation of the probe 102 for each acoustic data that is collected during the US scan. By knowing for each collected acoustic image the position and orientation of the probe 102, additional scan patterns can be planned to capture additional acoustic image data of a specific anatomical landmark of the eye that is identified in one of the images.

A fellow eye positioning/monitoring unit 114 is configured to perform at least one of: (i) monitoring the position and/or gaze direction of the fellow eye, namely the eye that is not being examined and remains wide open; (ii) providing guidance for the desired gaze direction of the fellow eye. The fellow eye positioning/monitoring unit 114 may include an optical sensor that is configured for imaging the fellow eye to derive its position and/or gaze direction. The conjugate eye movement allows to derive the position of the examined eye by knowing the position of the fellow eye. The optical sensor generates fellow eye positioning data FEPD and transmits it to the processing circuitry 108. The processing circuitry may use the fellow eye positioning data FEPD to conjugate between scan patterns and the position of the fellow eye. For example, if a specific scan requires the examined eye to gaze in a certain direction, when the processing circuitry recognizes that the fellow eye gazes in the desired direction, it executes the scan pattern. Furthermore, the fellow eye positioning/monitoring unit 114 may include a guiding unit for guiding the subject to turn its gaze towards a desired direction. The guiding unit may be audio-based, e.g. a speaker that outputs guidance for gaze direction and/or visual-based, e.g. an array of lights that is configured to turn on one or more specific lights in the array for guiding the subject to gaze on said turned on lights.

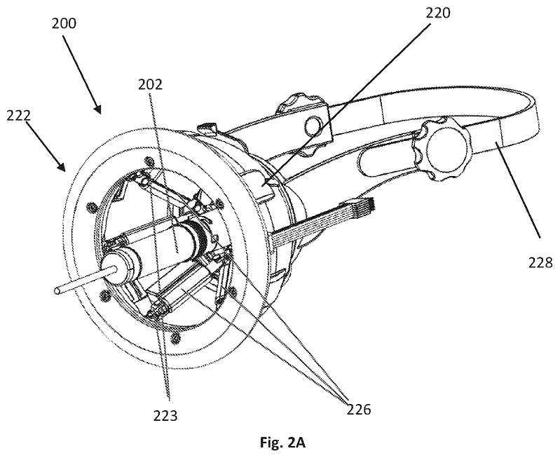

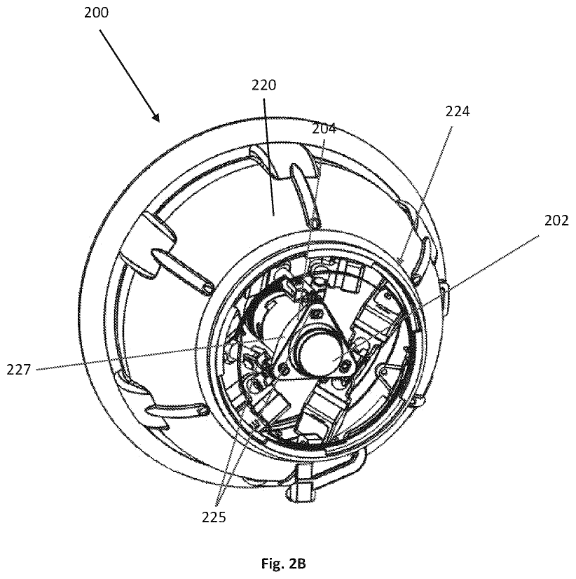

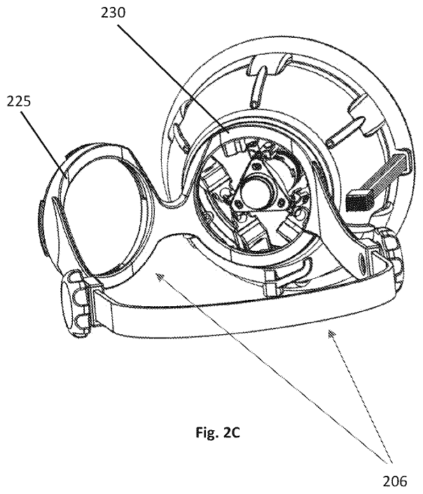

In the figures throughout the application, like elements of different figures were given similar reference numerals shifted by the number of hundreds corresponding to the number of the respective figure. For example, element 202 in FIGS. 2A-2C serves the same function as element 102 in FIGS. 1A-1B.

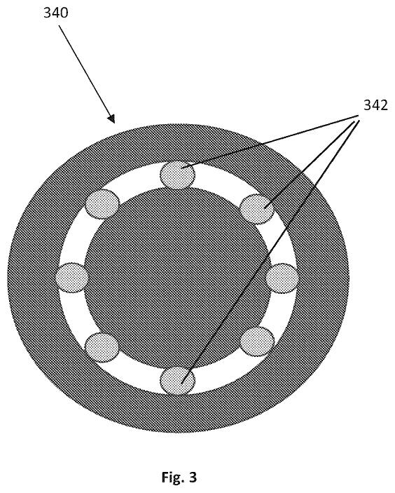

Reference is now made to FIGS. 2A-2C, which are perspective views of a non-limiting example of the system according to an embodiment of the present disclosure. FIG. 2A shows the system from its back side, FIG. 2B shows the front side of the examined eyed part and FIG. 2C shows the front side of the system. The system 200 includes a first housing 220 that extends between a back end 222 and a front end 224 and has a rounded longitudinal cross-section with a varying diameter. Generally, the diameter of the rounded longitudinal cross-section decreases towards the front end 224 of the housing. A US probe 202 is fixed to a probe-motioning unit 204 such that it is generally located along a central axis X of the housing 220, at least when it is at the default position. The US probe 202 to includes transducing surface 219 configured for emitting US signals and detecting their reflections. The transducing surface 219 spans a plane that generally overlaps with a plane spanned by the front end 224 of the housing. The probe-motioning unit 204 comprises a plurality of moving arms 226 that are fixed at a first end 223 to the housing 220 and at a second end 225 to a support platform 227 supporting the US probe 202. It is to be noted that the second end of the moving arms can be fixed directly to the US probe. By controlling the movement of the arms 226, movement in six degrees of freedom is obtained. This can be performed, for example, by a Stewart platform. A placement arrangement 206 of the system 200 comprises an adjustable strap 228 for adjusting the system over the subject's head to position the examined eye and the fellow eye at a desired position with respect to different elements of the system, e.g. the US probe aligned with the examined eye and an eye positioning/monitoring unit (not shown) aligned with the fellow eye. The position of the scanned eye is determined, among other optional tools, by monitoring the position of the fellow eye due to the conjugated movement of the eyes. Thus, the eye positioning/monitoring unit can be referred to as a fellow eye positioning/monitoring unit. As can be appreciated in FIG. 2C, the adjustable strap 228 is coupled to a frame 230 that fits over the front end 224 of the housing 220 and is configured to fit over the face of the user or more specifically over the eyes' orbits of the user. The frame 231 is constituted by an examined eye portion 233 that is coupled to the housing 220 including the US probe 202 and by a fellow eye portion 235 that is intended to fit over the fellow eye orbit of the user. In some embodiments, the system 200 may include a second housing (not shown) that comprises the eye positioning/monitoring unit that is being coupled to or integrated with the fellow eye portion 235. An example of the eye positioning/monitoring unit is exemplified in FIG. 3, which is a schematic illustration of a top view of a non-limiting embodiment of the eye positioning/monitoring unit. The eye positioning/monitoring unit 340 includes a plurality of light sources 342 arranged in a circle. The fellow eye of the user is intended to be positioned such that it generally faces the center of the circle. By activating a light source, the user is triggered to direct the gaze of the fellow eye towards the lit light source and due to the conjugated movement of the eyes, the examined eye also gazes the same direction. Another optional realization of the eye positioning/monitoring unit is by implementing in the system a speaker and the processing circuitry of the system is configured to operate the speaker to output guiding instructions for the gaze direction of the user in accordance with the scanning process and the requirements thereof. Thus, by the eye positioning/monitoring unit 342, the system can control the examined eye gazing direction as required according to the US scanning process. The placement arrangement 206 is configured for placing each of the eyes against the respective housing. Thus, the system generally has the shape of binocular or glasses. The front frame 230 of the housing 220 is designed for laying a part of the head. i.e. the surrounding portion of the eye, in particular the orbit. The adjustable strap 228 and the design of the front frame of the housing ensure that the examined eye and/or the fellow eye are placed at the desired position with respect to the elements of the system.

The system 200 includes a processing circuitry, i.e. a controller, that is in data communication with the probe 202 and the probe-motioning unit 204 to control the operation thereof. The processing circuitry may be housed within one of the housing of the system or is external thereto. The processing circuitry is configured for executing US scan patterns according to a selected pattern. The selected pattern may be a pre-set scan pattern that is carried out as a default as a first scan of a subject. During a US scan pattern acoustic data is collected and received in the processing circuitry 208. The acoustic data is analyzed to identify conditions of the eye. The identification of these conditions is performed based on machine learning techniques and the processing circuitry is pre-trained to identify a list of predetermined features. By analyzing the acoustic data, the processing circuitry classifies whether a condition exist or do not exist in the acoustic data, i.e. in an image data collected from the US scan. Furthermore, the processing circuitry analyzes the collected acoustic data to plan an additional US scan. The planning of the additional US scan is based on any one of the following: missing image data of anatomical landmark, questionable determination of existence of a condition, required image data of missing anatomical landmark, etc. A first US scan may provide the position of an anatomical landmark of the eye that serves as a perspective point for further scans, e.g. the location of the optic nerve may be used as a landmark for additional scans. The processing circuitry is configured to plan a subsequent scan for scanning anatomical landmarks of the eye and may use the location of the optic nerve to plan the pattern of the scan with respect to its location for obtaining the desired data. Each of the scans may be either A-scan that provides data on the length of the eye or B-scan that produces a cross-sectional view of the eye and the orbit, or a combination thereof. Measurements derived from the A-scan include, among others, spike height, regularity, reflectivity, and sound attenuation, while measurements derived from B-scan include visualization of the lesion, including anatomic location, shape, borders, and size.

1-25. (canceled)

26. A system for performing an automated ultrasound (US) scan of an eye of a subject, comprising:
a US transducer probe configured for generating ultrasonic signals and detecting reflections thereof;
a placement arrangement for allowing positioning of the eye of the subject at a desired position with respect to the US probe;
a probe-motioning unit configured to allow movement of the probe in at least three degrees of freedom;
a processing circuitry configured for controlling the movement of the probe-motioning unit and the operation of the US probe to execute at least a first US scan pattern through the eyelid of the subject or directly from the eye surface to collect at least a first US acoustic data indicative of one or more eye conditions of the subject.

27. The system of claim 26, wherein said first US scan pattern is a pre-set US scan pattern.

28. The system of claim 26, wherein the processing circuitry is configured to:
(i) analyze said first US acoustic data to identify one or more regions of interest;
(ii) plan a second US scan pattern and control the probe-motioning unit and the US probe for executing said second US scan for collecting a second US acoustic data indicative of said regions of interest.

29. The system of claim 26, wherein the processing circuitry is further configured for performing feature analysis on said at least first US acoustic data to identify one or more predefined conditions in the eye and/or the optic nerve of the subject;
wherein said feature analysis comprises scoring each of said predefined conditions and the identification of a condition is determined upon satisfying a certain a score condition.

30. The system of claim 26, wherein the processing circuitry is configured to analyze, segment and process the first US acoustic data, wherein the first US acoustic data comprises A or B scan images related to the position, shape, thickness or relative distance of ocular and intraocular structures, to their echogenicity or to the velocity of blood within the intraocular or the optic nerve vessels, spontaneously or as a result of calibrated pressure applied to the eye wall; and
wherein the processing circuitry is configured to extract from said at least first US acoustic data biometry parameters indicative of the eye structure of the subject.

31. The system of claim 26, wherein the processing circuitry is configured to control parameters of said US.

32. The system of claim 26, wherein the processing circuitry is configured to perform real-time image analysis of said at least first acoustic data and real-time adjusting the respective US scan pattern for collecting desired acoustic data.

33. The system of claim 26, wherein the placement arrangement comprises a fixation unit for fixedly association of the head of the subject to the US probe; and
wherein the fixation unit comprises fasteners for fastening the head of the unit.

34. The system of claim 26, being portable and designed to fit over a head of the subject.

35. The system of claim 26, wherein the US probe is fixed to the probe-motioning unit and move in correlation with the movement of the probe-motioning unit.

36. The system of claim 26, wherein the probe-motioning unit comprises a platform and the US probe is mounted thereon.

37. The system of claim 26, comprising a pressure sensor for measuring the applied pressure on the eyelid by the US probe;
wherein the processing circuitry is configured to receive the pressure measurement and adjust the applied pressure to be within a selected pressure range.

38. The system of claim 26, comprising a probe positioning sensor that is configured to monitor the spatial position of the US probe and transmit positioning data to the processing circuitry;
wherein the processing circuitry is configured to process the positioning data for monitoring and/or adjusting the movement of the probe-motioning unit for executing the desired first or second US scan pattern.

39. The system of claim 26, comprising a fellow eye positioning and/or monitoring unit that comprises at least one of:
(a) a light emitting device to guide the movement of the fellow eye thereby resulting in an alignment of the examined eye along a pre-defined or desired position;
(b) an optical sensor to monitor the position of the fellow eye that is indicative of the position of the examined eye.

40. The system of claim 26, comprising ocular movement guiding unit recognizable by the subject for guiding ocular movement of the subject in accordance with the executed US scan pattern.

41. The system of claim 40, wherein the ocular movement guiding unit comprises visual means, visible by the fellow eye of the subject while a US scan is performed by the system, for guiding the gaze of the fellow eye.

42. The system of claim 40, wherein the ocular movement guiding unit comprises a speaker device operable by the processing circuitry of the system to produce audible guiding instructions for ocular movement of the subject in accordance with the US scan pattern.

43. The system of claim 40, wherein the ocular movement guiding unit comprises a pressure generating component for applying localized pressure on subsequent regions of the face of the subject thereby guiding the movement of the eye being scanned or the fellow eye.

44. The system of claim 40, wherein the processing circuitry is configured to operate the ocular movement guiding unit in synchronization with the execution of any of the at least first US scan patterns.

45. The system of claim 26, comprising a safety switch for allowing immediate stop of the operation of the system.
